(19) United States
(12) Patent Application Publication
(10) Pub. No.: US 2016/0113579 A1
(43) Pub. Date: Apr. 28, 2016

(54) AUDIO OUTPUT APPARATUS, ELECTRONIC APPARATUS AND BODY FAT MEASURING METHOD

(71) Applicant: Samsung Electronics Co., Ltd., Gyeonggi-do (KR)

(72) Inventors: Kem Suk Seo, Gyeonggi-do (KR); Sung Soo Moon, Gyeonggi-do (KR); Bong Jae Rhee, Gyeonggi-do (KR); Sang Moon Lee, Gyeonggi-do (KR); Chang Su Lee, Gyeonggi-do (KR)

(21) Appl. No.: 14/920,787

(22) Filed: Oct. 22, 2015

(30) Foreign Application Priority Data
Oct. 22, 2014 (KR) 10-2014-0143165

(51) Int. Cl. A61B 5/00; H04R 1/10; H04R 1/02

(52) U.S. Cl. CPC A61B 5/6803; H04R 1/028; H04R 1/1041; A61B 5/4872; A61B 5/7278; A61B 5/0022; A61B 5/7405; H04R 2420/07; A61B 2560/0475

(57) ABSTRACT

A portable audio output apparatus, including an audio output module configured to be in contact with a user's body and to output an audio signal, and a body fat sensor configured to sense a body resistance value of the user through at least four body contact nodes, is provided. At least two of the nodes are placed at outer surface areas of the audio output apparatus that are out of contact with the body if the user wears the audio output apparatus.

### CROSS-REFERENCE TO RELATED APPLICATION AND CLAIM OF PRIORITY

The present application is related to and claims benefit under 35 U.S.C. §119(a) of a Korean patent application filed on Oct. 22, 2014 in the Korean Intellectual Property Office and assigned Serial number 10-2014-0143165, the entire disclosure of which is hereby incorporated by reference.

### TECHNICAL FIELD

The present disclosure relates to an audio output apparatus, an electronic apparatus, and a body fat measuring method with the apparatuses.

### BACKGROUND

With advancement of electronic technology, various types of electronic apparatuses have been steadily developed and supplied over the industry and the market. Evaluative growth of electronic apparatuses helps human lives to be more convenient and abundant day by day. Especially, portable electronic apparatuses, such as smartphones, laptop computers, tablet computers, wearable computers, and so on, are being widely popularized and extensively used.

As a wearable audio output apparatus, such as earphone or headphone, is inevitable to be in contact with a human body, it may be easily permissible to attach a biosensor to the audio output apparatus for the purpose of sensing a physical state of a user.

There is being developed the technology of monitoring a physical state of a user by attaching a heartbeat sensor to a body contact part of a wearable audio output apparatus such as headphone. However, since many nodes are needed to measure a body fat percentage of a user, it is impossible to accurately measure the body fat percentage through very restrictive body contact parts.

Moreover, a body fat percentage is highly relevant to a figure of a user and generally regarded as information which should be secured for personal privacy as like body weight. As a measured result from a general body fat measuring apparatus is output through a monitor, it may fail to secure personal privacy.

### SUMMARY

To address the above-discussed deficiencies, it is a primary object to provide at least the advantages described below. Accordingly, an aspect of the present disclosure is to provide an audio output apparatus capable of accurately measuring a body fat percentage of a user, an electronic apparatus and body fat measuring, method with the audio output apparatus.

In accordance with an aspect of the present disclosure, a portable audio output apparatus according to various embodiments of the present disclosure includes an audio output module configured to be in contact with a user's body and to output an audio signal, and a body fat sensor configured to sense a body resistance value of the user through at least four body contact nodes, wherein at least two of the nodes are placed at outer surface areas of the audio output apparatus that are out of contact with the body when the user wears the audio output apparatus.

In accordance with another aspect of the present disclosure, a portable electronic apparatus according to various embodiments of the present disclosure include a communication interface configured to communicate with an audio output apparatus having a body fat sensor, a control module configured to calculate a body fat percentage from a body resistance value that is received from the audio output apparatus; and a memory configured to store the body fat percentage, wherein if a new body fat percentage is calculated, the control module compares the new body fat percentage with a previous body data that is stored in the memory and controls the communication interface to transmit an audio signal, which is selected based on a result of the comparison, to the audio output apparatus.

In accordance with another aspect of the present disclosure, a body fat measuring method of a portable electronic apparatus according to various embodiments of the present disclosure include receiving a body resistance value from an audio output apparatus, calculating a body fat percentage based on the body resistance value, comparing the body fat percentage with a previous body fat percentage, selecting an audio signal based on a result of the comparison, and transmitting the audio signal to the audio output apparatus.

Other aspects, advantages, and salient features of the disclosure will become apparent to those skilled in the art from the following detailed description, which, taken in conjunction with the annexed drawings, discloses various embodiments of the present disclosure.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term. “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a configuration of a portable body fat measuring system according to various embodiments of the present disclosure;

FIG. 2 illustrates a configuration of an electronic apparatus according to various embodiments of the present disclosure;

FIG. 3 illustrates an example of an audio output apparatus according to various embodiments of the present disclosure;

FIGS. 4A, 4B, 5A, 5B, 6 and 7 illustrate positions of body fat sensors according to various embodiments of the present disclosure; and

FIG. 8 illustrates a body fat measuring method according to various embodiments of the present disclosure.

Throughout the drawings, it should be noted that like reference numbers are used to depict the same or similar elements, features, and structures.

### DETAILED DESCRIPTION

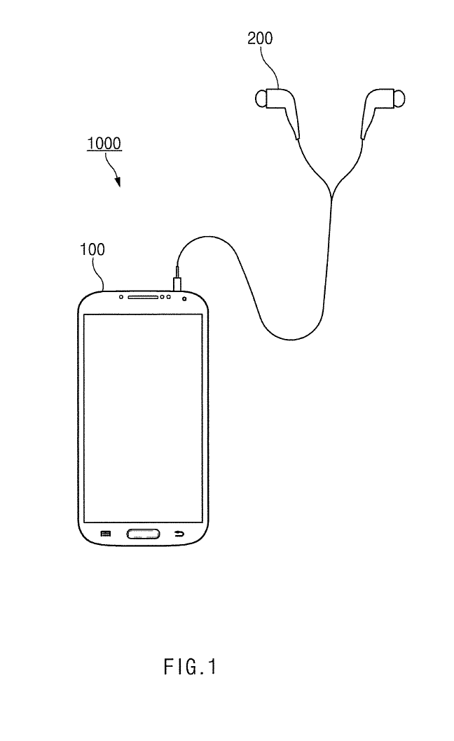

FIGS. 1 through 8, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged device. Hereinafter, various embodiments of the present disclosure will be described in conjunction with the accompanying drawings. Various embodiments described herein, however, may not he intentionally confined in specific embodiments, but should be construed as including diverse modifications, equivalents, and/or alternatives. With respect to the descriptions of the drawings, like reference numerals refer to like elements.

The terms “have,” “may have,” “include,” “may include,” “comprise,” or “may comprise” used herein indicate existence of corresponding features (e.g., numerical values, functions, operations, or components) but does not exclude other features.

As used herein, the terms “A or B,” “at least one of A or/and B,” or “one or more of A or/and B” may include all allowable combinations that are enumerated together. For example, the terms “A or B,” “at least one of A and B,” or “at least one of A or B” may indicate all cases of: (1) including at least one A, (2) including at least one B, or (3) including both at least one A, and at least one B.

As used herein, the terms such as “1st,” “2nd,” “first,” “second,” and the like may be used to qualify various elements regardless of their order and/or priority, simply differentiating one from another, but do not limit those elements thereto. For example, both a first user device and a second user device indicate different user devices. For example, a first element may be referred to as a second element and vice versa without departing from the scope of the present disclosure.

As used herein, if one element (e.g., a first element) is referred to as being “operatively or communicatively connected with/to” or “connected with/to” another element (e,g., a second element), it should be understood that the former may be directly coupled with the latter, or connected with the latter via an intervening element (e.g., a third element). Otherwise, it will be understood that if one element is referred to as being “directly coupled with/to” or “directly connected with/to” with another element, it may be understood that there is no intervening element (e.g., a third element) existing between them.

In the description or claims, the term “configured to” (or “set to”) may be changeable with other implicative meanings such as “suitable for”, “having the capacity to”, “designed to”, “adapted to”, “made to”, or “capable of”, and may not simply indicate “specifically designed to”. Alternatively, in some circumstances, a term “a device configured to” may indicate that the device “may do” something together with other devices or components. For instance, a term “a processor configured to (or set to) perform A, B, and C” may indicate a generic-purpose processor (e.g., CPU or application processor) capable of performing its relevant operations by executing one or more software or programs that is stored in an exclusive processor (e.g., embedded processor), which is prepared for the operations, or in a memory.

The terms used in this specification are just used to describe various embodiments of the present disclosure and may not, be intended to limit the scope of the present disclosure. The terms of a singular form may include plural forms unless otherwise specified. Unless otherwise defined herein, all the terms used herein, which include technical or scientific terms, may have the same meaning that is generally understood by a person skilled in the art. It will be further understood that terms, which are defined in a dictionary and commonly used, should also be interpreted as is customary in the relevantly related art and not in an idealized or overly formal detect unless expressly so defined herein in various embodiments of the present disclosure. In some cases, terms even defined in the specification may not be understood as excluding embodiments of the present disclosure.

An electronic device according to various embodiments of the present disclosure includes, for example, at least one of smartphones, tablet personal computers (tablet PC), mobile phones, video telephones, electronic book readers, desktop PCs, laptop PCs, netbook computers, workstations, servers, personal digital assistants (PDA), portable multimedia players (PMP), MP3 players, mobile medical devices, cameras, wearable devices (e.g., electronic glasses, or head-mounted devices (HMD), electronic apparel, electronic bracelets, electronic necklaces, electronic appoessories, electronic tattoos, smart mirrors, smart watches, and the like.

In some embodiments, an electronic device is a smart home appliance. The smart home appliance, for example, includes at least one of televisions (TV), digital versatile disc (DVD) players, audios, refrigerators, air conditioners, cleaners, ovens, microwave ovens, washing machines, air cleaners, set-top boxes, home automation control panels, security control panels, TV boxes (e.g., SAMSUNG HOMESYNC™, APPLE TV™, GOOGLE TV™, and the like), game consoles (e.g., XBOX™, PLAYSTATION™, and the like), electronic dictionaries, electronic keys, camcorders, electronic picture frames, and the like.

In certain embodiments, an electronic device includes at least one of diverse medical devices (e.g., portable medical measuring instruments (blood-sugar measuring instruments, heart-pulsation measuring instruments, blood-pressure measuring instruments, or body-temperature measuring instruments), magnetic resonance angiography (MRA) equipment, magnetic resonance imaging (MRI) equipment, computed tomography (CT) equipment, scanners, and ultrasonic devices), navigation device, global positioning system (GPS) receiver, event data recorder (EDR), flight data recorders (MR), vehicle infotainment devices, electronic equipment for vessels (e.g., navigation systems and gyrocompasses), avionics, security devices, head units for vehicles, industrial or home robots, automatic teller's machines (ATM) for financial agencies, points of sales (POS) for stores, and internet of things (e.g., electric bulbs, diverse sensors, electric or gas meter, spring cooler units, fire alarms, thermostats, road lamps, toasters, exercise implements, hot water tanks, boilers, and the like).

According to some embodiments, an electronic device includes at least one of parts of furniture or buildings/structures having communication functions, electronic boards, electronic-signature receiving devices, projectors, and diverse measuring instruments (e.g., water meters, electricity meters, gas meters, and wave meters) including metal cases. In various embodiments, an electronic device is one or more combinations of the above-mentioned devices. Electronic devices according to certain embodiments are flexible electronic devices. Additionally, electronic devices according to various embodiments of the present disclosure may not be restrictive to the above-mentioned devices, rather includes new electronic devices emerging by way of technical development.

Hereinafter, an electronic device according to various embodiments will be described in conjunction with the accompanying drawings. In description for various embodiments, the term “user” refers to a person using an electronic apparatus or an apparatus (e.g., an artificial intelligent electronic apparatus) using an electronic device.

FIG. 1 is a diagram illustrating a configuration of a portable body fat measuring system according to various embodiments of the present disclosure.

Referring to FIG. 1, a portable body fat measuring system 1000 includes an electronic apparatus 100 and an audio output apparatus 200.

Referring to FIG. 1, the electronic apparatus 100 and the audio output apparatus 200 communicate with each other in a wired manner through an audio interface of the electronic apparatus 100. According to various embodiments, the electronic apparatus 100 and the audio output apparatus 200 are connected through wireless communication technologies such as BLUETOOTH, NFC, and so on.

According to certain embodiments, the electronic apparatus 100 is implemented with a portable electronic apparatus such as smart phone, tablet PC, laptop PC, wearable devices (e.g., smart watch, smart glasses, etc.), and so on. According to certain embodiments, the audio output apparatus 200 is implemented with a portable audio output apparatus made in a wearable form by a user, such as earphone, headphone, and so on.

According to certain embodiments, the electronic apparatus 100 receives a body resistance value from the audio output apparatus 200 and calculates a body fat percentage of a user. The electronic apparatus 100 generates audio data based on the calculated body fat percentage. The electronic apparatus 100 transmits audio data to the audio output apparatus 200 and informs a user about a result of calculating a body fat percentage.

According to certain embodiments, the electronic apparatus 100 compares a calculated body fat percentage with a previous body fat percentage and selects a specific audio signal based on a result of the comparison. The electronic apparatus 100 informs a user of a body-fat measured result by transmitting a selected audio signal to the audio output apparatus 200.

According to certain embodiments, the audio output apparatus 200 senses a body resistance value of a user by using a body fat sensor. The audio output apparatus 200 transmits a measured body resistance value to the electronic apparatus 100. According to certain embodiments, the audio output apparatus 200 outputs an audio signal that is received from the electronic apparatus 200, and informs a user of a body-fat measured result.

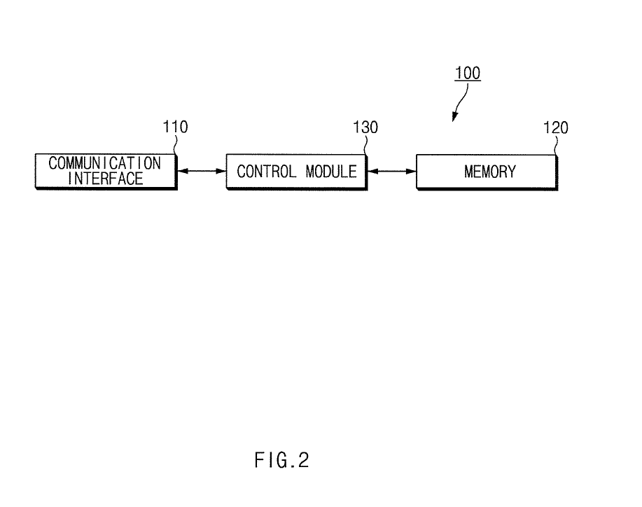

FIG. 2 is a block diagram illustrating a configuration of an electronic apparatus according to various embodiments of the present disclosure.

Referring to FIG. 2, the electronic apparatus 100 includes a communication interface 110, a memory 120, and a control module 130.

The communication interface 110 communicates with the audio output apparatus 200. According to certain embodiments, the communication interface 110 transmits an audio signal, such as voice or music, to the audio output apparatus 200. According to certain embodiments, the communication interface 110 receives a body resistance value from the audio output apparatus 200.

According to certain embodiments, the communication interface 110 includes an audio interface that is connected to the audio output apparatus 200 in a wired manner. According to certain embodiments, the audio interface transmits one of an audio signal or a body resistance value.

According to certain embodiments, the communication interface 110 includes a BLUETOOTH module that is wirelessly connected to the audio output apparatus 200. According to certain embodiments, the BLUETOOTH module coincidently transmits an audio signal and a body resistance value by using a plurality of channels prepared for signal transmission.

The memory 120 stores body fat percentages that are calculated by the control module 130. According to certain embodiments, the memory 120 stores calculated body fat percentages in the order of date and time. According to certain embodiments, the memory 120 stores an audio signal.

The control module 130 controls a general operation of the electronic apparatus 100. According to certain embodiments, if a body resistance value is received, the control module 130 calculates a body fat percentage from the body resistance value.

According to certain embodiments, if a body fat percentage is calculated, the control module 130 generates voice data that informs a user of the body fat percentage and controls the communications interface 110 to transmit the audio data to the audio output apparatus 200.

According to certain embodiments, the control module 130 stores a calculated body fat percentage in the memory 120. According to certain embodiments, if a body fat percentage is newly calculated, the control module 130 compares the new body fat percentage with a previous body fat percentage. The control module 130 selects an audio signal, which is to be transmitted to the audio output apparatus 200, based on a result of the comparison. For example, if a body fat percentage becomes lower than a previous body fat percentage, the control module 130 select a cheerful music. In contrast, if a body fat percentage becomes higher than a previous body fat percentage, the control module 130 selects a grave music or a warning sound. If an audio signal is selected, the control module 130 controls the communication interface 110 to transmit the selected audio signal to the audio output apparatus 200.

According to certain embodiments, the control module 130 controls the communication interface 110 to transmit an audio signal to the audio output apparatus 200 by using an audio interface. According to certain embodiments, if an audio signal is being transmitted through an audio interface, the control module 130 controls the communication interface 110 to interrupt the transmission of the audio signal and to transmit a new audio signal to the audio output apparatus 200.

According to certain embodiments, the control module 130 controls the communication interface 110 to transmit a selected audio signal by using a BLUETOOTH module. According to certain embodiments, if another audio signal is being transmitted through a BLUETOOTH module, the control module 130 controls the communication interface to transmit a new audio signal, which informs a body fat percentage, through a channel that is different from a channel through which the another audio signal is being transmitted.

According to certain embodiments, if an audio signal is being transmitted through a BLUETOOTH module, the control module 130 controls the communication interface 110 to mix and transmit the audio signal, which is being transmitted, and a new audio signal, which informs a body fat percentage. For example, the control module 130 performs mixing such that volume of a previously transmitted audio signal becomes smaller and, amplitude of an audio signal informing a body fat percentage becomes relatively larger.

A portable electronic apparatus according to various embodiments of the present disclosure includes a communication interface configured to communicate with an audio output apparatus having a body fat sensor, a control module configured to calculate a body fat percentage from a body resistance value that is received from the audio output apparatus; and a memory configured to store the body fat percentage, wherein if a new body fat percentage is calculated, the control module compares the new body fat percentage with a previous body data that is stored in the memory and controls the communication interface 110 to transmit an audio signal, which is selected based on a result of the comparison, to the audio output apparatus.

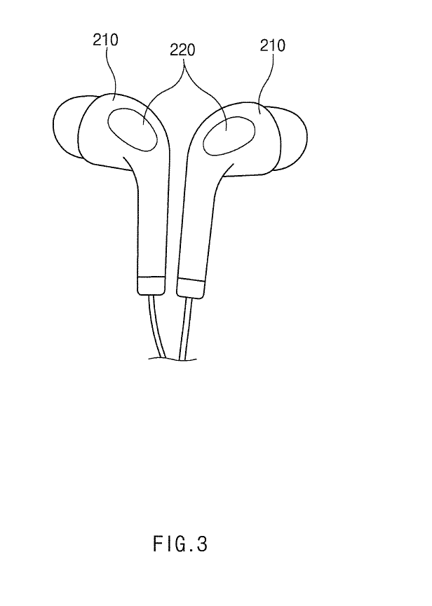

FIG. 3 is a diagram illustrating an example of an audio output apparatus according to various embodiments of the present disclosure.

Referring to FIG. 3, an audio output apparatus 200 includes an audio output module 210 and a sensor module 220.

The audio output module 210 outputs an audio signal that is received from the electronic apparatus 100. The audio output module 210 converts an audio signal, which is received from the electronic apparatus 100 in electrical form, into vibration and then transfers the vibration to a user. In the case that a user wears the audio output apparatus 200, it is permissible for at least a part of the audio output apparatus 200 to be in contact with a user's body (e.g., ear).

The body fat sensor 220 is in contact with a user's body and sense a body resistance value. According to certain embodiments, the body fat sensor 220 includes at least four body contact nodes. If a user is in contact with at least four body contact nodes with his/her body, the body fat sensor 220 measures a body resistance value through the four body contact nodes.

According to certain embodiments, the body fat sensor 220 is placed on the outer surface of the audio output apparatus 200. According to certain embodiments, at least two of the nodes of the body fat sensor 220 are placed on parts that are not in contact with a user's body in the outer surface of the audio output apparatus 200 when the user wears the audio output apparatus 200.

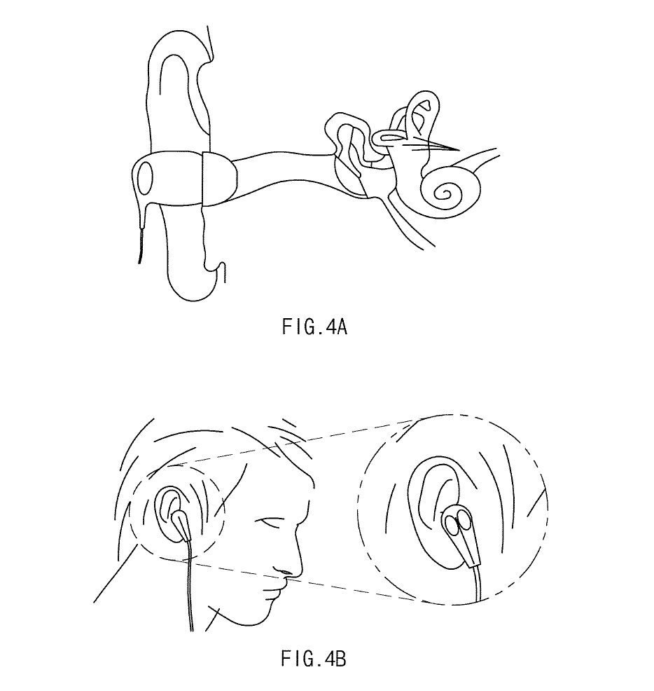

FIGS. 4A and 4B are diagrams illustrating a position of a body fat sensor according to various embodiments of the present disclosure.

FIGS. 4A and 4B illustrate a state of wearing an audio output apparatus 200 of earphone type. FIG. 4A illustrates a sectional view taken at the front of a user who is wearing the audio output apparatus 200, and FIG. 4B illustrates a side view of the user.

Referring to FIGS. 4A and 4B, at least a part of the audio output module 210 is inserted into a user's ear. Referring to FIGS. 4A and 4B, the body fat sensor 220 is placed at the opposite side of a direction in which the audio output module 210 is inserted into a user's ear.

According to certain embodiments, a part of nodes included in the body fat sensor 220 is placed at an outer surface area that is in contact with a user's body, and the other part of the nodes is placed at another outer surface area that is not in contact with the user's body.

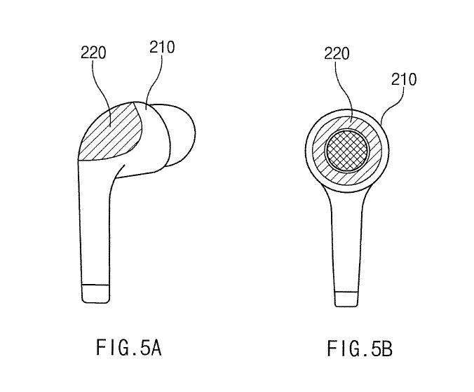

FIGS. 5A and 5B are diagrams illustrating positions of body fat sensors according to various embodiments of the present disclosure

FIGS. 5A and 5B illustrate views of an earphone-type audio output apparatus 200 at different angles. Referring to FIG. 5A, a node included in the body fat sensor 220 is placed at an outer surface area of the audio output module 210 that is not in contact with a user's body. Referring to FIG. 5B, a node included in the body fat sensor 220 is inserted into a user's ear by an outer surface area of the audio output module 210 and is placed at a body contact part of the audio output module 210.

According to certain embodiments, the audio output apparatus 200 includes a signal line 230 that is connected to an electronic apparatus 100 and that transfers a signal (e.g., a body resistance value or audio signal). According to certain embodiments, the audio output apparatus 200 is connected with the signal line 230 and includes an input module 240 that receives a user manipulation. According to certain embodiments, at least a part of body contact nodes included in the body fat sensor is placed at the input module 240.

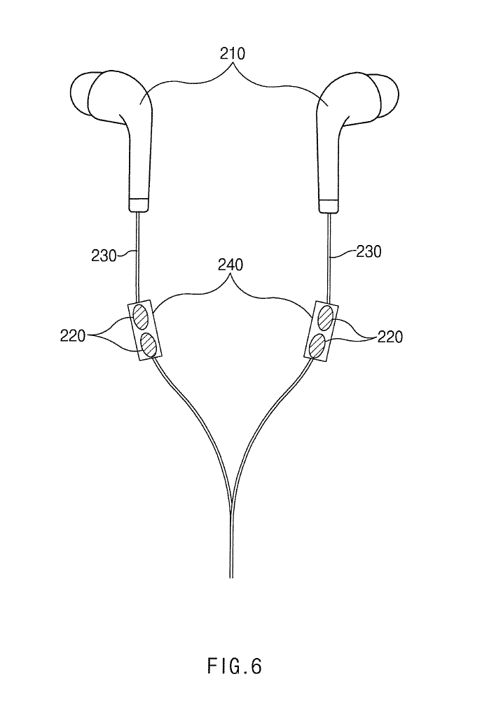

FIG. 6 is a diagram illustrating positions of body fat sensors according to various embodiments of the present disclosure.

Referring to FIG. 6, an ear-phone type audio output apparatus 200 is illustrated therein. In the audio output apparatus 200, two audio output modules 210 are connected respectively with signal lines 230. The signal lines 230 are connected with the two input modules 240 that receive a user manipulation. Referring to FIG. 6, a plurality of nodes included in the body fat sensor 220 is placed on the outer surface of the input module 240. For example, a plurality of nodes is placed on the outer surface of the input module 240. Alternatively, a part of the plural nodes is placed on a body contact area of the outer surface of the audio output module 210 while the other part of the plural nodes is placed on the outer surface of the input module 240.

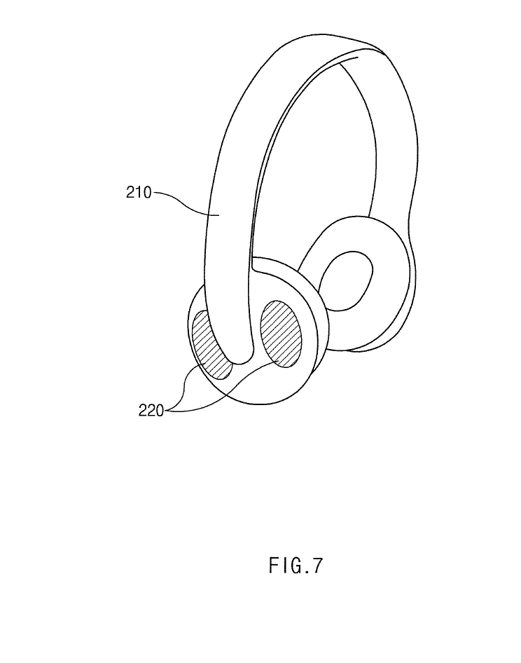

FIG. 7 illustrates an example of an audio output apparatus according to various embodiments of the present disclosure.

FIG. 7 illustrates a headphone-type audio output apparatus 200. Referring to FIG. 7, a node of a body fat sensor 220 is placed on an outward surface area of a headphone-type audio output module 210 that is not in contact with a user's body when the user wears the audio output apparatus 200.

Although not illustrated in FIGS. 3 to 7, according to certain embodiments, an audio output apparatus 200 include a BLUETOOTH module (not shown). According to certain embodiments, a BLUETOOTH module is connected with an electronic apparatus 100 and transmits a body resistance value or audio signal to the electronic apparatus 100.

According to certain embodiments, an audio output apparatus 200 include a switch (not shown) that performs a switching operation to connect a signal line 230 with one of an audio output module 210 or a body fat sensor.

According to certain embodiments, a switch (not shown) is implemented with a button switch. According to certain embodiments, if a switch (not shown) is implemented with a button switch, the switch is connected with a body fat sensor 220 and is placed under the body fat sensor. For example, a switch (not shown) is connected with a part of a plurality of nodes of a body fat sensor 220. For example, if a user presses at least a part of a plurality of nodes of a body fact sensor 220 to measure body resistance, a switch (not shown) connects a signal line 230 with the body fat sensor 220.

According to certain embodiments, a switch. (not shown) is an electrically operating switch. For example, if a user is in contact with at least a part of a plurality of nodes of a body fat sensor 220 and the body fat sensor 220 senses a body resistance value, a switch (not shown) connects a signal line 230 with the body fact sensor 220.

A portable audio output apparatus according to various embodiments of the present disclosure includes an audio output module configured to be in contact with a user's body and to output an audio signal, and a body fat sensor configured to sense a body resistance value of the user through at least four body contact nodes, wherein at least two of the nodes are placed at outer surface areas of the audio output apparatus that are out of contact with the body when the user wears the audio output apparatus.

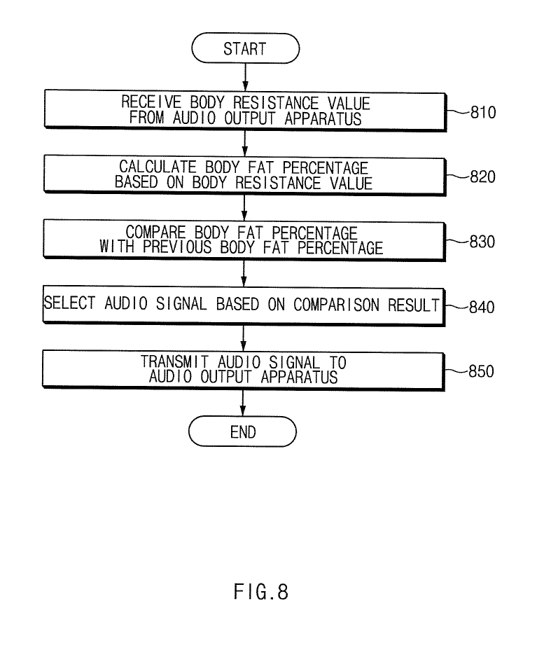

FIG. 8 is a flow chart showing a body fat measuring method according to various embodiments of the present disclosure. The flow chart shown in FIG. 8 is composed of operations executed in the electronic apparatus 100 of FIG. 2. Therefore, even for the contents omitted below, the description about the electronic apparatus 100 of FIG. 2 is referred to the flow chart of FIG. 8.

In operation 810, the electronic apparatus 100 receives a body resistance value from an audio output apparatus.

In operation 820, the electronic apparatus 100 calculates a body fat percentage based on the body resistance value.

In operation 830, the electronic apparatus 100 compares the body fat percentage with a previous body fat percentage. According to certain embodiments, the electronic apparatus 100 stores a calculated body fat percentage in a memory. According to certain embodiments, if a new body fat percentage is calculated, the electronic apparatus 100 compares the new body fat percentage with a previous body fat percentage that is stored in the memory 120.

In operation 840, the electronic apparatus 100 selects an audio signal, which is to be transmitted to the audio output apparatus 200 based on a result of the comparison. For example, if a body fat percentage becomes lower than a previous body fat percentage, the electronic apparatus 100 selects a cheerful music. On the other hand, if a body fat percentage becomes higher than a previous body fat percentage, the electronic apparatus 100 selects a grave music or warning sound.

In operation 850, the electronic apparatus 100 transmits an audio signal to the audio output apparatus 200. According to certain embodiments, the electronic apparatus 100 transmits an audio signal through an audio interface. According to certain embodiments, if an audio signal is being transmitted through an audio interface, the electronic apparatus 100 interrupts the transmission of the audio signal and transmits a new audio signal that informs a body fat percentage. According to certain embodiments, the electronic apparatus 100 transmits a selected audio signal through a BLUETOOTH module. According to certain embodiments, if another audio signal is being transmitted through a BLUETOOTH module, the electronic apparatus 100 transmits an audio signal, which informs a body fat percentage, to a channel different from the channel transmitting the another audio signal.

According to certain embodiments, if an audio signal is being transmitted through a BLUETOOTH module, the electronic apparatus 100 mixes the transmitted audio signal with a new audio signal that informs a body fat percentage. For example, it is allowable to mix a previously transmitted audio signal, which is controlled to be smaller in volume, and a body-fat informing audio signal that is controlled to be relatively larger in amplitude.

A body fat measuring method of a portable electronic apparatus according to various embodiments of the present disclosure includes receiving a body resistance value from an audio output apparatus, calculating a body fat percentage based on the body resistance value, comparing the body fat percentage with a previous body fat percentage, selecting an audio signal based on a result of the comparison, and transmitting the audio signal to the audio output apparatus.

A body fat measuring method of an electronic apparatus according to various embodiments of the present disclosure is implemented by a program executable in the electronic apparatus. Such a program is stored in various kinds of recording media and is used for the body fat measurement.

In detail, program codes for performing the aforementioned methodological features is stored in various kinds of nonvolatile recording media such as flash memory, Read-Only Memory (ROM), Erasable Programmable ROM (EPROM), Electrically Erasable and Programmable ROM (EEPROM), hard disk, removable disk, memory card, USB memory, CD-ROM, and so on.

According to various embodiments of the present disclosure, it is allowable to accurately measure a body fat percentage without limitations on time and space by using a portable apparatus. Additionally, since a result of measuring body fat is informed by voice or music through an audio output apparatus, it is possible to secure personal privacy.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A portable audio output apparatus comprising:
an audio output module configured to:
contact a user's body, and
output an audio signal; and
a body fat sensor configured to sense a body resistance value of the user through at least four body contact nodes,
wherein at least two of the nodes are placed at outer surface areas of the audio output apparatus that are out of contact with the body when the user wears the portable audio output apparatus.

2. The portable audio output apparatus of claim 1, wherein a part of the nodes is placed at an outer surface area of the audio output apparatus that is in contact with the body when the user wears the portable audio output apparatus, and the other part of the nodes is placed at an outer surface area of the audio output apparatus that is out of contact with the body when the user wears the portable audio output apparatus.

3. The portable audio output apparatus of claim 1, further comprising:
a signal line connected with an electronic apparatus and configured to transmit a signal; and
an input module connected with the signal line and configured to receive a user manipulation,
wherein at least a part of the nodes is placed at the input module.

4. The portable audio output apparatus of claim 1, further comprising:
a signal line connected to an electronic apparatus and configured to transmit a signal; and
a switch configured to switch the signal line to be connected with one of the audio output module or the body fat sensor.

5. The portable audio output apparatus of claim 4, wherein the switch is a button switch, and
wherein if the switch is pressed, the signal line is connected with the body fat sensor.

6. The portable audio output apparatus of claim 4, wherein the signal comprises a body resistance value or an audio signal.

7. A portable electronic apparatus comprising:
a communication interface configured to communicate with an audio output apparatus including a body fat sensor;
a control module configured to calculate a body fat percentage from a body resistance value that is received from the audio output apparatus; and
a memory configured to store the body fat percentage,
wherein if a new body fat percentage is calculated, the control module is further configured to compare the new body fat percentage with a previous body data that is stored in the memory and control the communication interface to transmit an audio signal, which is selected based on a result of the comparison, to the audio output apparatus.

8. The portable electronic apparatus of claim 7, wherein the communication interface comprises a BLUETOOTH module, and
wherein, if another audio signal is being transmitted through the BLUETOOTH module, the control module is further configured to control the communication interface to transmit the selected audio signal to a channel that is different from a channel that transmits the another audio signal.

9. The portable electronic apparatus of claim 7, wherein the communication interface comprises a BLUETOOTH module, and
wherein, if another audio signal is being transmitted through the BLUETOOTH module, the control module is further configured to mix the another audio signal and a body-fat informing audio signal.

10. The portable electronic apparatus of claim 7, wherein the communication interface comprises an audio interface, and
wherein, if another audio signal is being transmitted through the audio interface, the control module is further configured to control the communication interface to interrupt transmission of the another audio signal and transmit the selected audio signal.

11. The portable electronic apparatus of claim 7, wherein the audio signal comprises first kind of sound if the body fat percentage becomes lower than the previous body at percentage.

12. The portable electronic apparatus of claim 7, wherein the audio signal comprises second kind of sound if the body fat percentage becomes higher than the previous body fat percentage.

13. The portable electronic apparatus of claim 7, wherein the audio signal includes the body fat percentage.

14. A body fat measuring method of a portable electronic apparatus, the method comprising:
receiving a body resistance value from an audio output apparatus;
calculating a body fat percentage based on the body resistance value;
comparing the body fat percentage with a previous body fat percentage;
selecting an audio signal based on a result of the comparison; and
transmitting the audio signal to the audio output apparatus.

15. The body fat measuring method of claim 14, wherein transmitting the audio signal to the audio output apparatus comprises:
if another audio signal is being transmitted through a BLUETOOTH module, transmitting the selected audio signal to a BLUETOOTH channel that is different from a BLUETOOTH channel that transmits the another audio signal.

16. The body fat measuring method of claim 14, wherein transmitting the audio signal to the audio output apparatus comprises:
if another audio signal is being transmitted through a BLUETOOTH module, mixing the another audio signal and a body-fat informing audio signal.

17. The body fat measuring method of claim 14, wherein transmitting the audio signal to the audio output apparatus comprises if another audio signal is being transmitted through an audio interface, interrupting the transmission of the another audio signal and transmitting the selected audio signal.

18. The body fat measuring method of claim 14, wherein selecting an audio signal based on a result of the comparison comprises, if the body fat percentage becomes lower than the previous body fat percentage, selecting a first kind of sound.

19. The body fat measuring method of claim 14, wherein selecting an audio signal based on a result of the comparison comprises, if the body fat percentage becomes higher than the previous body fat percentage, selecting a second kind of sound.

20. The body fat measuring method of claim 14, wherein the selected audio signal includes the body fat percentage.
